(19) United States
(12) Patent Application Publication
(10) Pub. No.: US 2009/0024017 A1
(43) Pub. Date: Jan. 22, 2009

(54) Electrophysiological sensor, weak electrical signal conditioning circuit and method for controlling said circuit

(75) Inventors: Giulio Ruffini, Barcelona (ES); Stephen Dunne, Barcelona (ES); Esteve Farres, Barcelona (ES)

Correspondence Address: RATNERPRESTIA, P.O. BOX 980, VALLEY FORGE, PA, 19482, US

(73) Assignee: STARLAB BARCELONA, SL, Barcelona (ES)

(21) Appl. No.: 12/107,392

(22) Filed: Apr. 22, 2008

Related U.S. Application Data
(63) Continuation-in-part of application No. PCT/ES2007/000390, filed on Jun. 29, 2007.

(30) Foreign Application Priority Data
Jul. 19, 2006 (ES) P 200601997

(51) Int. Cl. A61B 5/05

(52) U.S. Cl. 600/395

(57) ABSTRACT

An electrophysiological sensor, weak electrical signal conditioning circuit and method for controlling the circuit as provided. The sensor includes rigid filiform conducting nanostructures connected to a conducting substrate and operable to penetrate an organic tissue. The circuit includes an instrumentation amplifier with an input connected to a first electrode in contact with a first area of a medium, and a second input; a voltage generating device connected to an electrode in contact with a second area of the medium for applying a continuous reference signal to it; a compensator, electrically insulated from the device, for compensating the direct current offsets of a weak electrical signal received by the first electrode, generating a signal with a reference voltage with a value which can be modified by a control system, and supplying it to the second input. A method is also provided for controlling the circuit.

### FIELD OF THE INVENTION

The present invention generally relates, in a first aspect, to an electrophysiological sensor, i.e., an electrode assembly for electrophysiological applications—and particularly to a sensor adapted for electrophysiological applications which do not require conducting substances acting as an interface between the sensor and the signal taking area.

The invention likewise relates to an electrophysiological sensor operating in dry conditions particularly applicable to transmitting weak electrical signals (such as biological potential or biopotential signals) with low noise, through the skin, based on nanotechnology.

The present invention also generally relates, in a second aspect, to a weak electrical signal, generally biological potential signal, conditioning circuit, and, in a third aspect, to a method for controlling such circuit, and particularly to a circuit and a method adapted for conditioning weak electrical signals coming from a medium, compensating possible interferences and direct current offsets experienced by such signals without inducing an additional current flow through the medium.

The invention is particularly applicable to obtaining and conditioning biopotential electrical signals from any part of the body of a patient, such as those obtained by means of electroencephalograms (EEG), electrocardiograms (ECG), electro-oculograms (EOG) or electromyograms (EMG).

### BACKGROUND OF THE INVENTION

The demand for electrophysiological sensors applied on the skin is currently increasing in modern clinics and in biomedical applications.

Patent application EP-A-1596929 describes a medical device comprising an electrode incorporating a material based on carbon nanotubes and a method for manufacturing the device.

Carbon nanotubes were discovered in approximately 1991 and from then on numerous and very interesting applications such as power storage for batteries, capacitors, etc. have been found for them. The previously mentioned patent application starts from the knowledge of these nanotubes for constructing a medical device. Said medical device is used for perceiving and communicating electrical stimuli in a way similar to that considered in the present invention, but in that patent application it is necessary to apply, in at least one portion of the surface of the medical device, an adhesive layer formed by a conductive polymer for adhering the nanotubes to at least one portion of the electrode. This layer is not necessary in the sensor of the present invention, being able to use techniques such as CVD (Chemical Vapor Deposition) for the direct growth of the nanotubes on a conducting substrate in the device.

The medical device described in patent application EP-A-1596929 does not describe nor suggest that the nanotubes arranged on a portion of the electrode are configured and/or adapted for directly penetrating the skin, it must be deduced therefrom that its application will be the conventional one by means of placing an interface layer between the electrode and the skin, generally a gel type interface layer.

The need to apply an interface layer between the electrode or sensor and the skin or another organic tissue involves some drawbacks: it is necessary to invest a long time preparing the skin and the device (in the order of a few minutes) for each intervention. The gel-skin interface is not a stable interface, which adds noise (electrode-skin, electrode-polymer or gel, gel-skin) to the measurements taken, disrupting its reliability. The conductive gel can further undergo modifications such as drying out (at least to a certain extent) during the process and therefore adding even more noise to the involved information. It is likewise necessary to act on the skin on which the device will be placed by slightly scratching or scraping the epidermal surface in order to unify it and ensure the conductivity with the consequent discomfort for the patient and operating time.

Due to the foregoing, it seems to be necessary to offer an alternative to the state of the art alleviating the discomfort generated in the patient, saving time for the operator, and reducing the disturbances and errors derived from the noise added to the measurements. It is therefore of interest to provide a sensor improving the previously indicated current deficiencies.

The electrophysiological sensor proposed by the first aspect of the present invention provides a particular solution to this need and unlike the device described in the mentioned patent application EP-A-1596929, in said alternative sensor it will be possible to dispense with the mentioned substrate-nanotube adhesive layer, furthermore adding a different operation based on penetrating the skin by the nanotubes, as is described below.

In addition, the circuitry currently used for measuring biopotential signals obtained by means of electrodes or electrophysiological sensors is based on the differential amplification and on the filters. High-gain amplification is carried out by means of a differential amplifier with a high input impedance, a high common-mode rejection ratio (CMRR) and a gain which is adjustable.

An instrumentation amplifier is a closed-loop gain unit which has a differential input and a single output with respect to a reference terminal. The impedances of the two input terminals are generally balanced and have high values, typically 10⁹ K, or greater. The input polarization currents must also be low, normally between 1 nA and 50 nA. As occurs with operational amplifiers, the output impedance is very low, nominally a few milliohms, at low frequencies.

Instrumentation amplifiers are a low-pass filter, in which the bandwidth for a unitary gain for a small signal is typically between 500 kHz and 4 MHz. A gain increase reduces the bandwidth but its response is very flat in the range of biopotential signals (up to a few kHz). For this reason, active low-pass filters are required for improving the frequency range of interest.

An ideal low-pass filter would completely eliminate the signals above the cutoff frequency and would allow the signals below it (within the passband) to perfectly pass. Several undertakings are carried out in the actual filters in order to attempt to approximate the ideal case. Several types of filters are optimized in order to obtain a flat gain response within the passband, others sacrifice gain variation (ripple) in the passband in order to obtain a more sudden drop in the edge of the passband, whereas others sacrifice both, the flat response and the drop ratio, in order to favor the reliability in the pulsatile response.

After the analog signal conditioning, the signal can be digitized with an analog-to-digital converter, or ADC. The Nyquist criterion requires that the sampling frequency be at least two times the highest frequency contained in the signal, or the information on the signal will be lost. If the sampling frequency is less than twice the maximum frequency of the analog signal, a phenomenon known as aliasing or overlapping will occur. It is important to point out that if an input filter is not arranged in the input of the ideal sampler, any frequency component (either a signal or noise) falling outside the Nyquist bandwidth will be overlapped, i.e. will undergo aliasing effects. For this reason a filter is used in almost all the ADC sampling applications for eliminating these unwanted signals.

The main problem in biopotential measurements are the interferences, including the dominant noise of the line frequency of 50 or 60 Hz. In known proposals of weak electrical signals conditioning circuits, specifically biopotential signals obtained by means of a series of electrodes, the active electrodes include a follower close to each of them for intermediately storing the signal of the electrode, such that virtually all interference problems associated with the differential and high impedances of each electrode are eliminated.

Respective examples of the state of the art with regard to ECG systems with their corresponding instrumentation amplifiers AD8220 and INA326 respectively, appear on page 2 of the bulletin “Amplifier ICs Volume 6, Issue 1” of “Analog Devices” and on page 17 of the document “Information for Medical Applications” from “Real World Signal Processing” from Texas Instruments (SLYB108).

The slight imbalances in the lengths and the contacts of the electrodes cause the common-mode signal to be offset in direct current, which forms the main limitation of the differential amplifier of the instrumentation amplifier. The right leg exciter circuit (DRL) attempts to reduce this limitation by applying a voltage close to the common-mode voltage in a voltage supplying reference electrode or DRL electrode. Other alternating current (AC) coupling techniques could be applied for changing the reference voltage of the instrumentation amplifier. The circuit shown in FIG. 2 of the document “Heart-Rate and EKG Monitor Using the MSP430FG439” from Texas Instruments (SLAA280) shows an example of said AC coupling techniques, in which the digital-to-analog converter DAC1 shown therein provides a mean supply voltage level as polarization voltage for the amplifier chain.

The lower limit of the noise level in the bioelectrical measurements is determined by the thermal noise of the impedance of the electrodes. Consequently, in order to achieve noise levels in the range of microvolts, the impedance of the electrode, including that existing between the electrode and the skin of the patient, must be less than 100 kΩ.

There are several patent documents describing different systems and methods for carrying out biopotential signal measurements. Several of said documents are set forth below.

Patent document EP1631189A1 discloses a system for carrying out biopotential signal measurements, in which a probe is used with an electrode located adjacent to the patient (in contact or without contact therewith). The use of a ground voltage, the voltage of a second electrode in contact with the patient or the voltage of a second electrode incorporated in the actual probe as the reference voltage of the differential amplifier to which the electrode is connected is proposed. The probe incorporating the electrode can include electronic circuitry with amplifiers, filtering and gain steps, batteries, components for transmitting through cable or wirelessly or components for recording data for a subsequent transmission. The probe includes a conductor separated a fixed distance with respect to the electrode adjacent to the patient for the purpose of insulating it against interference signals, for example coming from the amplifier included in the actual probe.

In addition, U.S. Pat. No. 5,876,351A1 proposes a portable modular device for carrying out electrocardiograms, which for several of its embodiments comprises amplifiers, each of them with a first input connected to each of several electrodes, previous to the instrumentation amplifier, and with a second input connected to a common reference voltage which can be that of the DRL (Wilson) or GND electrode, selected by means of a multiplexer.

U.S. Pat. No. 5,392,785A1 proposes an instrumentation amplifier applied to reducing common-mode voltage in ECG and EEG measurements by means of the application by a compensation circuit a compensation voltage representative of said common-mode voltage in different manners, some with a third DRL electrode and others without this third electrode but with a feedback path for high frequencies between a capacitor included in a compensation circuit and the capacity existing between the patient and the ground, or chassis, through said ground or chassis. The input of the amplifier of the compensation circuit is connected to the outputs of the differential amplifiers of the signals of the electrodes, for the purpose of monitoring the common-mode voltage received through the electrodes.

U.S. Pat. No. 5,713,365A1 proposes dispensing with the DRL electrode by maintaining a good rejection to the electric noise, directly feeding back the common-mode voltage detected in the output of the differential amplifiers of each electrode, in the inputs thereof, for the purpose of obtaining a portable device more than that of improving the performance of the systems which do inject a reference signal through the DRL electrode.

In all the mentioned system proposals including DRL circuits there is an electrical connection between the latter and the circuit conditioning the signals coming from the electrodes, since the voltage applied to the DRL electrode corresponds to the common-mode voltage detected in the differential amplifiers connected to each electrode, which makes the fluctuations in the electrical signals provided by the electrodes and the possible artifacts, or interfering signals, cause changes in the voltage to be applied to the DRL electrode, which results in the occurrence of a current injection in the patient through the DRL electrode-skin-tissue interface, altering the balance in the potentials of the half-cells.

The differential amplifiers of each electrode in the known proposals share one and the same reference voltage.

None of the mentioned proposals describes the electric separation of the DRL circuit from the circuit conditioning the electrical signals coming from the electrodes for the purpose of preventing the mentioned unwanted current injection in the medium, nor that of using individual reference voltages for each electrode for the purpose of comparing each of them with one of the signals supplied by each of the receiver electrodes in respective instrumentation or differential amplifiers.

### DESCRIPTION OF THE INVENTION

The present invention relates, in a first aspect, to a electrophysiological electrode assembly or sensor which is basically integrated by an electrode for the skin which can be used in multiple applications such as: electroencephalograms (EEG), electrocardiograms (ECG), electro-oculography (EOG), or electromyography (EMG) among others. The sensor is based on technology with nanostructures and is applied in the transmission of weak electrical signals, with low noise, through the skin. It consists of an assembly formed by a structure of carbon nanotubes (CNT) with a particular arrangement and configuration especially favorable for electrophysiological applications.

More specifically, the proposed electrophysiological sensor is of the type comprising a plurality of conducting nanostructures which can transmit a weak electrical signal captured from the skin or from another part of an organic tissue to a transmitter means formed for example by an electrical connector which continues in a conductor or wiring and is characterized by integrating a structure of multiple carbon nanotubes fixed to a suitable conducting support substrate, said nanotubes emerging from said substrate in the form of substantially rigid and filiform elements, like needles. The fixing does not require a polymer layer for adhering the nanotubes. These rigid and filiform nanostructures are thus chemically connected at one end to said conducting substrate linked to an electrical connector and are operable to at least partially penetrate said organic tissue or skin at their free end like needles, without needing any intermediate gel or interface, i.e., the electrophysiological sensor operates in dry conditions on the skin which must not have been previously subjected to a previous preparation treatment. The contact and partial penetration of the nanotubes in the Stratum Corneum (outer layer of the epidermis with a thickness of 10 to 20 μm and forming a resistive medium) allow a stable electrical contact with low impedance and noise.

In a first embodiment the electrophysiological sensor of the invention comprises an envelopment casing housing said connector and which has associated to an outer wall the mentioned conducting substrate supporting the nanostructures.

According to a second preferred embodiment it has been provided that the sensor includes a local amplifier in association with said connector.

In an even more improved third version the sensor additionally includes a circuit for a treatment (pre-processing or processing) or at least partial adaptation of the weak electrical signals captured from the skin.

In an even more improved fourth version, the incorporation of an electronic circuit for transmitting the data captured by the sensor, by radiofrequency, to a remote management point has been provided.

The transmission and control of the electronic circuit associated to sensor is carried out wirelessly being able to couple, if necessary, other digital communication devices, as can be the case of a USB communications port.

The sensor will generally incorporate a digital electronic circuit carrying out signal conditioning, automatic gain control, automatic drift compensation, digitization and digital filtering functions.

These functions allow considerably reducing the interferences of the signal and form a substantial improvement with respect to the state of the art.

Another function of the mentioned electronic circuit is the possibility of compressing the digitized signals, prior to transmitting said signals to the signal storage and display devices, thus allowing to reduce the rate of information to be transmitted and the consumption of the device.

Differential modulation techniques such as for example Continuously Variable Slope Delta-modulation (CVSD), widely used for voice transmission in Bluetooth devices, are used for compressing the digitized signal.

The measurement and arrangement of the mentioned nanotubes incorporated in the body of the sensor, projecting therefrom grouped like a brush, is such that they can penetrate the stratum corneum, the last layer of the skin, thus offering a better reception of the electrical signal.

This new arrangement for the capturing means of the sensor allows:
1. Preventing the need to scrape the skin of the area chosen for capturing data, thus reducing the time used in the measurement and the discomfort caused to the patient.
2. Dispensing with an interface substance or gel, thus eliminating the noise introduced in the measurement caused by said interface substance or gel and also decreasing the time used in the measurement.

In other words, by using the electrophysiological sensor proposed by the present invention, a preparation of the skin before or after transmitting electrical signals through the skin will not required.

Said nanotubes can likewise be incorporated in a mold placed or fixed in different supports such as a garment, a pillow or a mattress, being able to go unnoticed by the patient and not interfering in the measurements taken. Furthermore, the actual tissues will also be able to have integrated communication infrastructures.

An electrophysiological sensor, such as that described herein can be used in communication in the body area by using weak and therefore safe electrical signals. In other words, using the inside of the body as a conductor for transferring information to the so-called Body network area, including communications with implants.

According to the first aspect of the present invention, the use of a technology of carbon nanotubes is highly interesting due to the fact that said nanotubes are extremely small, thus increasing health safety by decreasing the risk of infection. The nanotubes used are good conductors, inert and extremely hard and consistent.

According to an embodiment, a reasonable value for the resistance of the assembly of carbon nanotubes has to be less than 100 kΩ; given that the impedance of a multiple wall carbon nanotube, which are those used herein, is 1000 kΩ, it is sufficient for a few to penetrate the skin or establish a low impedance electrical contact by means of another mechanism (capacitance). From this data and carrying out the appropriate calculations the conclusion is reached that the sensor proposed by the first aspect of the present invention will consists of at least 20 nanotubes considering that all of them have fall contact with the skin.

The present invention also relates to using an electrophysiological sensor as that proposed by the first aspect of the invention, in EEG, ECG, EMG, EOG, or for brain-machine interface applications, biometry applications or systems for detecting fatigue and hypovigilance, as well as to using said sensor for monitoring the wakefulness or sleep state in an individual.

With regard to the circuitry used for conditioning weak signals, generally biopotential signals, in view of the state of the art the main drawbacks of which have been set forth above, it seems to be necessary to offer an alternative which enables solving said drawbacks, and which particularly prevents altering the balance in the potentials of the half-cells occurring in the conventional proposals upon injecting a current into a medium, generally a patient, through a reference electrode, such as that described above as a DRL electrode.

To that end the present invention relates in a second aspect to a weak electrical signal conditioning circuit, of the type comprising:
at least one adjustable-gain instrumentation amplifier with a high input impedance, with:
   a first input in connection with a first receiver electrode in contact with a first area of a medium for receiving at least one of said weak electrical signals coming from said medium, and
   a second input in connection with a reference voltage,
a reference voltage generating device in connection with a voltage supplying electrode in contact with a second area of said medium for applying a reference electrical signal to it so that the voltage existing in the medium has a value substantially equal to that of said electrical signal generated by said reference voltage generating device, and
compensation means for compensating at least the direct current offsets experienced by at least said weak electrical signal received by a receiver electrode.

Unlike the conventional proposals the circuit proposed by the second aspect of the invention is adapted for applying to the voltage supplying electrode, by means of said reference voltage generating device, a continuous electrical signal with a fixed value, the mentioned compensation means being insulated electrically with respect to the constant reference voltage generating device and substantially with respect to the voltage supplying electrode for assuring that there is no current flow through the medium as a result of the action of the compensation means, contrary to what occurred with the previously described conventional conditioning circuits, in which the fluctuations in the electrical signals provided by the receiver electrodes and the possible artifacts, or interfering signals, caused a current injection into the medium through the reference electrode, altering the balance in the potentials of the half-cells.

In the circuit proposed by the second aspect of the invention the mentioned compensation means are electrically independent from the reference voltage generating device, and comprise:
a voltage generating device connected to the second input of the instrumentation amplifier, for generating and supplying to it an electrical signal with a reference voltage, for compensating at least the unwanted direct current offsets experienced by the weak electrical signal received by the first receiver electrode, and
a control system in connection with said voltage generating device, and adapted for controlling it for the purpose of modifying the value of said reference voltage.

Said control system is generally connected with the output of said instrumentation amplifier for controlling the voltage generating device according to the output signal of the instrumentation amplifier.

For an embodiment the mentioned compensation means are adapted for, for the purpose of complementing said compensation carried out by means of applying said reference voltage, supplying the instrumentation amplifier, through a direct current offset adjustment input, with a direct current compensation adjustment signal at the output of the instrumentation amplifier representative of a variable voltage value determined according to the direct current offset to be compensated experienced by the weak electrical signal received by the first receiver electrode.

The compensation means are also adapted for compensating alternating interfering signals by means of supplying said electrical signal with a reference voltage and/or said adjustment signal, to said second input and to said direct current offset adjustment input of the instrumentation amplifier, respectively.

In the present application weak electrical signals are understood to be signals with low amplitude and/or coming from sources with high output impedances. The AC voltage values of such signals are typically less than 10 mV and can have DC direct components greater than the AC signal.

For a preferred embodiment the conditioning circuit proposed by the second aspect of the invention is applied to conditioning biopotential measurement signals on any part of the body of a patient, such as those obtained by means of electroencephalograms (EEG), electrocardiograms (ECG), electro-oculograms (EOG) or electromyograms (EMG), said medium being a patient, for the purpose of achieving a minimum current flow therethrough.

For said preferred embodiment the voltage supplying electrode is in contact with a contact area of said patient, forming with said constant voltage generating device a right leg circuit DRL for the purpose of achieving that the voltage of the patient is substantially equal to the voltage supplied by the constant voltage generating device.

A third aspect of the present invention relates to a method for controlling a weak electrical signal conditioning circuit, of the type which comprises:
receiving at least one of said weak electrical signals coming from a medium, through a first receiver electrode in contact with a first area of said medium,
sending said received weak electrical signal to a first input of an adjustable-gain instrumentation amplifier with a high input impedance,
applying a reference voltage to a second input of said instrumentation amplifier,
applying a reference electrical signal to a voltage supplying electrode in contact with a second area of said medium so that the voltage existing in the medium has a value substantially equal to that of said reference electrical signal applied to said voltage supplying electrode, and
compensating at least the direct current offsets experienced by said weak electrical signal received by the first receiver electrode.

The method proposed by the third aspect of the invention is characterized in that:
said reference electrical signal applied to voltage supplying electrode is a continuous electrical signal with a fixed value, independent from the common-mode signal of said instrumentation amplifier,
and in that said compensation of at least the direct current offsets experienced by said weak electrical signal received by the first receiver electrode, is automatically carried out by means of compensation means substantially electrically insulated with respect to said voltage supplying electrode for assuring that there is no current flow through the medium as a result of the action of the compensation means.

For an embodiment the method comprises carrying out said compensation of the direct current offsets experienced by said weak electrical signal received by the first receiver electrode by means of applying said reference voltage and modifying its value in a controlled manner.

The proposed method comprises carrying out said compensation or compensations for compensating the common-mode voltage variations of said instrumentation amplifier.

The method proposed by the third aspect of the invention is applied to conditioning biopotential measurement signals, said medium being a patient, for the purpose of achieving a minimum current flow through the medium.

Although it is not limited to it, the method proposed by the third aspect of the invention is applied to controlling a conditioning circuit according to the second aspect of the invention.

In addition, the circuit proposed by the second aspect of the invention can be applied to conditioning signals obtained by different types of electrodes (wet, dry, etc.), but for a preferred embodiment the electrodes comprise one or more electrophysiological sensors such as that proposed by the first aspect of the invention, in which case the digital electronic circuit described for several embodiments of the sensor proposed by the first aspect, which carries out signal conditioning, automatic gain control, automatic drift compensation, digitization and digital filtering functions, is for the present preferred embodiment the conditioning circuit proposed by the second aspect of the invention.

The interferences of the biopotential signals are considerably reduced, and the offsets experienced by such signals are compensated by means of implementing said preferred embodiment, using the high performance of both the electrophysiological sensor proposed by the first aspect, and the conditioning circuit proposed by the second aspect.

### BRIEF DESCRIPTION OF THE DRAWINGS

The previous and other advantages and features will be more fully understood from the following detailed description of several embodiments with reference to the attached drawings, which must be considered in an illustrative and non-limiting manner, in which:

FIG. 1 shows an assembly of carbon nanotubes (CNT) according to the first aspect of the invention applied directly to the skin, it being observed that said nanostructure penetrates the stratum corneum, the last layer of the skin and also observing the cover of the sensor and the installation for transmitting the signal schematically;

FIG. 2 shows a second embodiment similar to the first in which an amplifier installed inside the cover of the assembly has been added to the sensor;

FIG. 3 shows an electrical diagram or equivalent circuit representative of the operation of the sensor described by the first aspect of the present invention, the resistance of the sensor, the properties of the interface and other variables being indicated in this depiction;

FIG. 4 is a schematic depiction of the conditioning circuit proposed by the second aspect of the invention applied to conditioning biopotential signals of a patient, specifically ECG signals, for an embodiment for which the signals come from a single receiver electrode;

FIG. 5 is a view similar to FIG. 4, but for an embodiment for which the conditioning circuit is applied to conditioning signals coming from two receiver electrodes,

FIG. 6 shows the method proposed by the third aspect of the invention, for an embodiment, by means of a flow chart,

FIGS. 7a to 7e are different views of a support supporting the circuit proposed by the second aspect of the invention and the electrodes connected thereto applied in the head of a patient, for the embodiment of an encephalogram, and

FIG. 8 is a perspective view of a casing or case housing part of the conditioning circuit proposed by the second aspect of the invention.

### DETAILED DESCRIPTION OF SEVERAL EMBODIMENTS

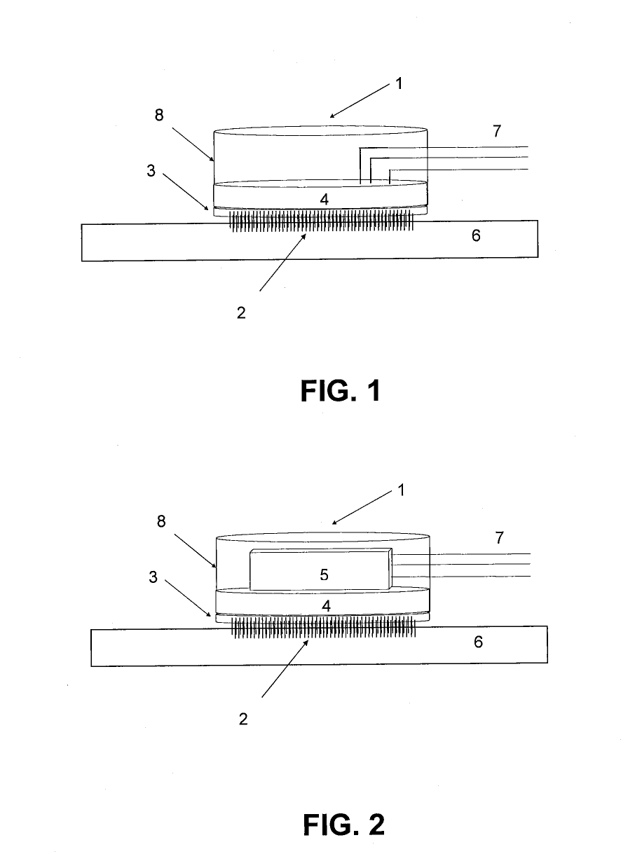

The electrophysiological sensor 1 according to the first aspect of the invention is integrated by a casing 8 housing an assembly of carbon nanotubes (CNT) 2 supported directly on a conducting substrate 3 from which they emerge like very fine rigid needles, suitable for being able to directly come into contact with the skin 6 without applying an electrolytic gel and at least partially penetrate the stratum corneum, the outermost layer of the skin, as observed in FIG. 1. FIG. 1 has also detailed an electrical connector 4 which is used as a bridge between the conducting substrate 3 and an installation or at least one wiring 7 for transporting the signal to a remote point.

With the electrophysiological sensor 1 presented by the first aspect of the present invention provided with a structure of inert (i.e., non-polarizable) carbon nanotubes 2, in the form of rigid filiform elements grouped like a brush which can penetrate said outer layer of the skin, a transmission of electrical signals carrying information is achieved, from an organic tissue, with less noise with respect to the sensors of the state of the art and without needing an intermediate interface layer. Said structure of carbon nanotubes (CNT) 2 is further designed so that it penetrates very little, without coming into contact with the nerve cells, thus preventing any sensation of pain in the patient or possible transmission of an infection.

Advantageously the carbon nanotubes 2 used have multiple walls whereby their conductivity is increased and the capture and transmission of the biological potential signals is favored.

The mentioned multiple wall carbon nanotubes 2 have been obtained by growing directly in the substrate 4, which is made of highly doped silicon or titanium.

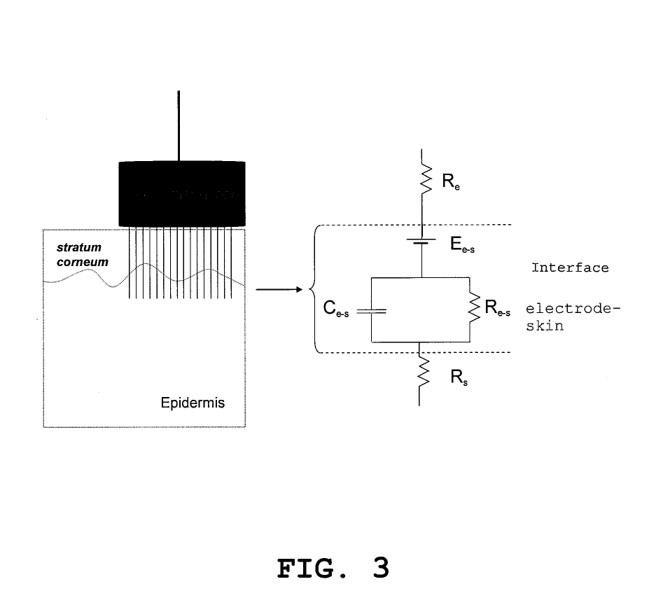

In another embodiment shown in FIG. 2 the sensor 1 additionally includes a signal amplification system 5 integrated within the actual casing 8 in order to decrease the noise in the measurements. In this case the previously described arrangement of elements is reproduced again, there is an interface of CNT 2 in the form of bristles of a brush in contact with the outermost layer of the skin 6 (slightly penetrating therein) and there is also an installation or transport means 7, such as a wiring for transporting the electrical signal, which this time will be connected directly to the amplifier device 5.

The sensor of the invention can be formed such that it can be directly incorporated into different substrates, such as garments, pillows, mattresses or others. In a particular embodiment it has been provided that said conducting substrate is integrated in a material forming part of a surface of said garment or other article providing a substrate.

Taking into account that the electrophysiological sensor of the invention operates as a biological electrode such as a transducer converting an ionic current into an electrical current, it has been provided that in a possible embodiment the sensor of the invention also use a suitable coating for facilitating the Red-Ox reaction in the interface area and for such purpose the mentioned nanotubes 2 are at least partially coated (in the region of their tips), with a suitable coating and particularly with an Ag—AgCl coating.

In another implementation of the invention it has been provided that the described electrophysiological sensor further houses an electronic circuit for processing and treating the signal, and a radio-frequency transmitter.

FIG. 3 shows a simulation by means of a equivalent circuit of the performance of the electrophysiological sensor of the invention, in relation to an ionic signal captured by the sensor.

Unlike the performance of a similar sensor of the state of the art in which there will be at least two RC couplings, with their corresponding associated output and input resistances, corresponding respectively to a first gel-skin interface and to a second gel-electrode interface, in the proposal of the present invention said RC coupling is limited to the skin-sensor coupling, a considerable reduction in the noise introduced in the captured weak signal being derived therefrom due to the smaller charge accumulations in the interface layers, especially upon avoiding the gel-skin interface which is known to provide a component of instability most probably due to the diffusion of the gel in the stratum corneum which is a dynamic non-homogeneous process.

The substitution of known electrophysiological electrodes for EEG, ECG, EMG, EOG, or for brain-machine interface applications, as well as the use of said sensor for communications between devices in the human body (external or implants) using the inside of the body as a conducting means, must be emphasized Among the applications for the electrophysiological sensor of the invention.

Other applications of the described electrophysiological sensor are in the area of research on sleep and particularly for monitoring the wakefulness or sleep state in an individual or for fatigue studies and in the area of biometry based on EEG and ECG.

The invention also relates to the use of an electrophysiological sensor according to the first aspect of the invention in EEG, ECG, EMG, EOG, or for brain-machine interface applications, biometry applications or systems for detecting fatigue and hypovigilance, as well as to the use of said sensor for monitoring the wakefulness or sleep state in an individual.

A person skilled in the art will be able to carry out variations and modifications on the embodiments indicated up to this point and introduce other elements into the capturing chain of the sensor, known in themselves, without departing from the scope of the present invention as it is defined by the attached claims.

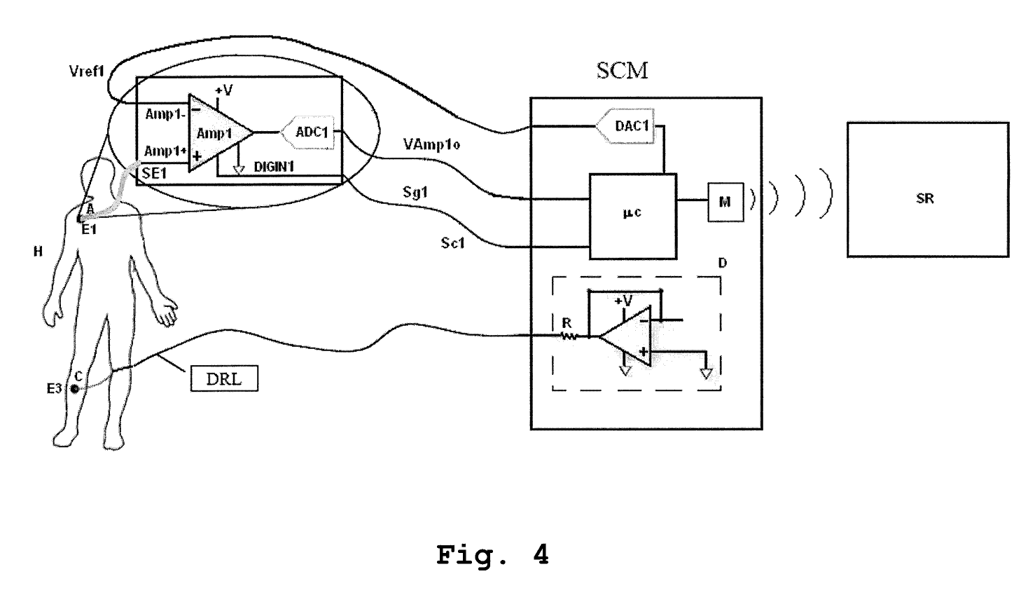

With reference to FIG. 4, it shows the conditioning circuit proposed by the second aspect of the invention for an embodiment for which it is applied to conditioning biopotential signals representative of an ECG conducted on a patient H.

The circuit of FIG. 4 comprises:
an adjustable-gain instrumentation amplifier Amp1 with a high input impedance, with:
   a first input Amp1+ in connection with a first receiver electrode E1 in contact with a first area A of said patient H (area arranged in the upper right torso of the patient H) for receiving a weak electrical signal SE1 therefrom, and
   a second input Amp1− in connection with a reference voltage Vref1,
a reference voltage generating device D in connection with a voltage supplying electrode E3 in contact with a second area C of said patient H, in this case the right let thereof, for applying a reference electrical signal to it so that the voltage existing in the medium, in this case a patient H, has a value substantially equal to that of said electrical signal generated by said reference voltage generating device D, which form a right leg circuit DRL with the electrode E3
compensation means for compensating the direct current offsets experienced by said weak electrical signal SE1 received by the first receiver electrode E1.

As has been previously stated the circuit proposed by the second aspect of the invention is adapted for applying to the voltage supplying electrode E3, by means of the reference voltage generating device D, a continuous electrical signal with a fixed value, said reference voltage generating device D being a constant voltage generating device D.

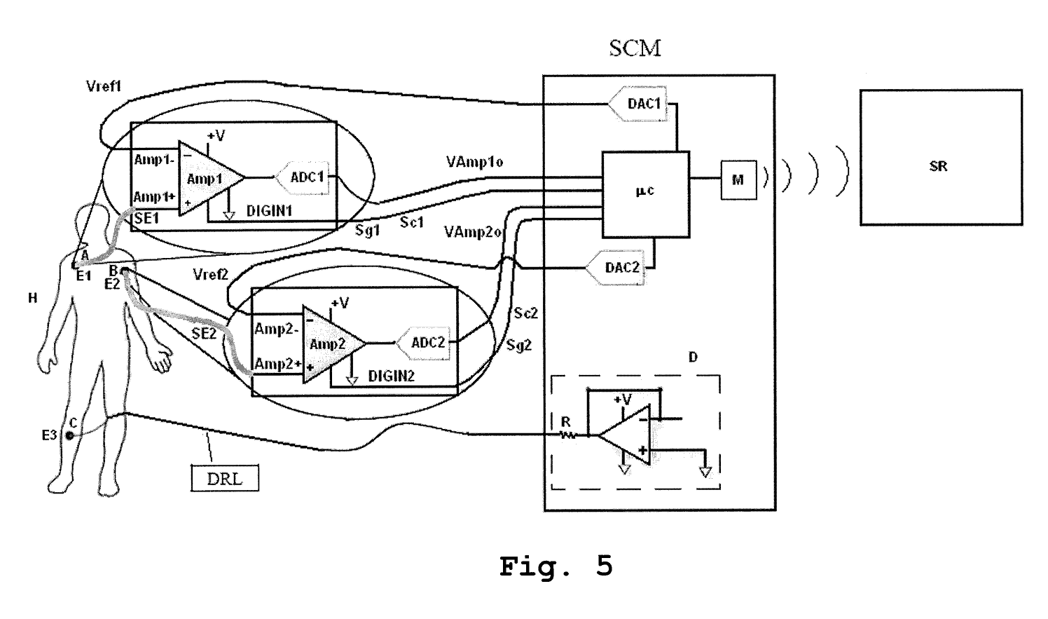

The constant voltage generating device D is formed, for the embodiments shown by FIGS. 4 and 5, by an optional amplifier with current limitation in order to comply with international recommendations, such that it is assured that the voltage supplied to the electrode E3 is very stable, said voltage being a precision voltage reference (precision band interval reference).

Since the compensation means are insulated electrically with respect to the constant voltage generating device D and substantially with respect to the voltage supplying electrode E3 (between E1 and E3 there is only the impedance of the patient H, which is very high) it is assured that there is no current flow through the medium H as a result of the action of the compensation means, contrary to conventional proposals in which said current flow causing changes in the potentials of the half-cells did occur.

The compensation means of the circuit proposed by the second aspect of the invention comprise, for the embodiment shown by FIG. 4:
a voltage generating device, which is a digital-to-analog converter DAC1 connected to the second input Amp1− of the instrumentation amplifier Amp1, for generating and supplying to it an electrical signal with a reference voltage Vref1 through said second input Amp1−, for the unwanted direct current offsets experienced by the weak electrical signal SE1 received by the first receiver electrode E1, and
a control system in connection with said voltage generating device DAC1, and adapted for controlling it for the purpose of modifying the value of said reference voltage Vref1.

As has been previously stated, the mentioned compensation means are adapted for supplying the instrumentation amplifier Amp1, through a direct current offset adjustment input DIGIN1, with a direct current compensation adjustment signal Sc1 at the output of the instrumentation amplifier Amp1 representative of a variable voltage value determined according to the direct current offset to be compensated experienced by said weak electrical signal SE1 received by the first receiver electrode E1.

Likewise the compensation means of the circuit proposed by the second aspect of the invention are also adapted to compensate alternating interfering signals by means of supplying said electrical signal with a reference voltage Vref1 and/or said adjustment signal Sc1, according to the necessary compensation.

The compensation means are adapted for, according to the ratio between the output signal of the instrumentation amplifier Amp1 and the dynamic range thereof:
adjusting said gain of the instrumentation amplifier Amp1 by means of sending a gain adjustment signal Sg1 to a gain adjustment input DIGIN1 thereof, and/or
carrying out said modification of the value of said reference voltage Vref1 to be applied to the second input Amp1− of the instrumentation amplifier Amp1, and/or
modifying the value of said variable voltage, and therefore said adjustment signal Sc1 representative thereof to be applied to said direct current offset adjustment input DIGIN1 of the instrumentation amplifier Amp1.

For the embodiments shown by FIGS. 4 and 5, said gain adjustment input and said direct current offset adjustment input are one and the same input DIGIN1 for programming the instrumentation amplifier Amp1, said adjustment signals Sg1, Sc1 being digital signals.

The mentioned control system is formed by a microcontroller μc (or by a logic circuit for other embodiments not shown), forming part of a local electronic system SCM which also includes said voltage generating devices D, DAC1, said control system being connected to the output of the instrumentation amplifier Amp1 once digitized by a digital-to-analog converter ADC1, for monitoring it and operating accordingly, said operation including the mentioned control of the digital-to-analog converter DAC1 for modifying the value of the reference voltage Vref1.

Continuing with FIG. 4, it also shows how the microcontroller μc comprises:
an output connected to DAC1 the output of which is connected to the second input Amp1− of the first instrumentation amplifier Amp1, for sending it the electrical signal with a reference voltage Vref1 after its conversion to an analog format, and
another output connected to an adjustment input DIGIN1 of the instrumentation amplifier Amp1 for supplying it with said adjustment signal Sc1, Sc2, Sg1, Sg2 in digital format.

FIG. 5 shows another embodiment similar to that shown by FIG. 4 but in which the conditioning circuit is applied to conditioning biopotential signals coming from two electrodes E1 and E2.

In said circuit shown by FIG. 5, it comprises a second adjustable-gain instrumentation amplifier Amp2 with a high input impedance, with:
a first input Amp2+ in connection with a second receiver electrode E2 in contact with a third area B of the patient H (in this case an area arranged in the upper left torso of the patient H), for receiving another one of said weak electrical signals SE2, and
a second input Amp2− in connection with a second reference voltage Vref2.

The compensation means illustrated in FIG. 5 are also adapted for compensating the direct current offsets (and where appropriate alternating current offsets) experienced by said weak electrical signal SE2 received by the second receiver electrode E2, and comprise a second voltage generating device DAC2 connected to the second input Amp2− of the second instrumentation amplifier Amp2, for generating and supplying to it an electrical signal with a second reference voltage Vref2, for carrying out said compensation.

As is seen in FIG. 5, the control system comprises the mentioned microcontroller μc also connected with said second voltage generating device DAC2, which is another digital-to-analog converter, and adapted for controlling it for the purpose of modifying the value of the second reference voltage Vref2 applied to the second input Amp2− of the second instrumentation amplifier Amp2.

The compensation means are adapted for compensating direct current offsets experienced by the weak electrical signal SE2 received by said second electrode E2 and alternating interfering signals, operating in a manner similar to how they operate with the differential amplifier Amp1, including the supply of a respective direct current compensation adjustment signal Sc2 and the sending of a gain adjustment signal Sg2 to an adjustment input DIGIN2 of the same Amp2.

The local control system SCM shown in FIG. 5 includes both the microcontroller μc and the constant voltage generating device D, as well as the two digital-to-analog converters DAC1 and DAC2.

The microcontroller μc of FIG. 5 comprises:
first and second outputs respectively connected to the first and second digital-to-analog converters DAC1, DAC2, and
third and fourth outputs respectively connected to the adjustment inputs DIGIN1, DIGIN2 of the instrumentation amplifiers Amp1, Amp2, for supplying them with said adjustment signals Sc1, Sc2, Sg1, Sg2 in digital format.

The polarization voltages of the amplifiers Amp1, Amp2 and of the operational amplifier of the generating device D, and indicated as +V in FIGS. 4 and 5 come from batteries (not shown) included in the local electronic system SCM, such that some of the interferences caused by the mains voltage when said circuits are supplied by the mains supply are prevented.

A suitable instrumentation amplifier for being used as Amp1 and Amp2 is for example the programmable-gain instrumentation amplifier AD8555, although the circuit shown by FIGS. 4 and 5 is not limited to any specific instrumentation amplifier, provided that it is has a programmable gain and has one or more gain programming and direct current offset compensation inputs, such as the inputs DIGIN1 and DIGIN2 shown.

It is necessary to point out that the conditioning circuit of FIGS. 4 and 5 has been shown in a simplified and schematic manner, including the most important elements forming part thereof, but the inclusion of other elements (such as filters) that are common in this type of circuits (some of which have been mentioned in the “State of the Art” section) is also contemplated by the present invention.

In fact the actual instrumentation amplifiers Amp1, Amp2, such as the mentioned AD8555, include a circuit for suppressing radio frequency interferences with a low-pass filter with a bandwidth of a few kHz.

A low-pass (RC) filter (not shown) is used before the digitalization, which filter allows the signals within the passband of the filter to pass through while at the same time it limits the bandwidth of the signals which are outside the passband, thus reducing the possible noise in the input signals of the analog-to-digital converters ADC1, ADC2, and therefore obtaining digital signals VAmp1o, VAmp2o representative of interference-free analog signals.

It is necessary to point out that some of the components or elements of the local electronic system SCM have also not been shown so that FIGS. 4 and 5 are clearer when schematically illustrating the main acting components of the circuit proposed by the second aspect of the invention. One of said not shown elements is a low noise linear regulator (such as ADP3331) which reduces the noise of the analog output signals of the SCM, i.e., Vref1, Vref2, and the reference voltage of electrode E3 of the DRL circuit.

As is schematically shown in FIGS. 4 and 5, the local electronic system SCM comprises a communications module M adapted for wirelessly communicating with a remote control system SR, preferably in a two-way manner.

For an embodiment, said remote control system SR is adapted for wirelessly receiving, from the local electronic system SCM, the digital values representative of the output signal VAmp1o, VAmp2o of the instrumentation amplifier (FIG. 4) or amplifiers (FIG. 5) Amp1, Amp2, and for analyzing them.

For an embodiment, the remote control system SR is adapted for carrying out, automatically or if necessary with the intervention of an operator (for example for choosing a control program to be applied), at least part of the gain adjustments of the instrumentation amplifiers Amp1, Amp2, and/or the modification of the values of the reference voltages Vref1, Vref2, and/or the modification of the adjustment signals Sc1, Sc2, and for carrying out the corresponding sendings of the digital values of the adjustment signals Sc1, Sc2, Sg1, Sg2 and/or of reference voltages Vref1, Vref2 to the local electronic system SCM.

For another embodiment, the local electronic system SCM is adapted for carrying out the gain adjustments of the instrumentation amplifiers Amp1, Amp2, and/or the modification of the value of the reference voltages Vref1, Vref2 and/or the modification of the adjustment signals Sc1, Sc2.

Such remote control system SR is, for an embodiment, a computerized system in connection with display means, such as a screen, for displaying the output signals VAmp1o, VAmp2o, i.e., for the case of an ECG, the signals representative thereof.

The mentioned remote computerized system SR preferably comprises a series of both input and output peripherals in order to enable its use by an operator for example for the mentioned embodiment in which part of the adjustments are carried out by the remote system SR.

The remote system SR obviously also has a communications module (not shown) which is internal or external (for example connected to a USB port) and which can wirelessly communicate with the communications module M of the local electronic system SCM with the same technology and protocols (for example Zigbee).

The main objective of using wireless communication is eliminating possible signal interferences with the mains frequency (50 or 60 Hz) caused by the use of cables. This is so due to the fact that said wireless interface or communication eliminates the main stray capacitance between the body of the patient and ground which occurred in the mentioned cables of conventional proposals. Said wireless communication obviously also provides great autonomy to the patient which the wiring does not allow.

For an embodiment, the instrumentation amplifiers Amp1, Amp2, associated circuitry, and in general the local electronic system SCM are supported by a support C which also supports all or part of the electrodes E1, E2, E3, together with the instrumentation amplifiers Amp1, Amp2 and associated circuitry ADC1, ADC2, filters (not shown), etc.

The fact that the signals of the receiver electrodes E1, E2 are amplified and digitized in situ, i.e., with the instrumentation amplifiers Amp1, Amp2, and other associated circuitry, arranged very close to the electrodes, considerably eliminates the interfering noise which conventionally occurs when the active electrodes are far from the amplifying steps, due to the high degree of rejection to the common-mode of the instrumentation amplifiers it eliminates the mains frequency noise common to the two electrodes E1, E2, and to the absence of cables between the electrodes E1, E2 and the amplification electronics. After the digitization in situ the digitalized output signal VAmp1o, VAmp2o (see FIGS. 4 and 5) has no interference problems.

The reference voltage of the supplying electrode E3 of the DRL circuit has a high immunity to noise, due to the fact that it comes from a dedicated noise cancellation circuit, which in FIGS. 4 and 5 is formed by a voltage follower based on an operational amplifier.

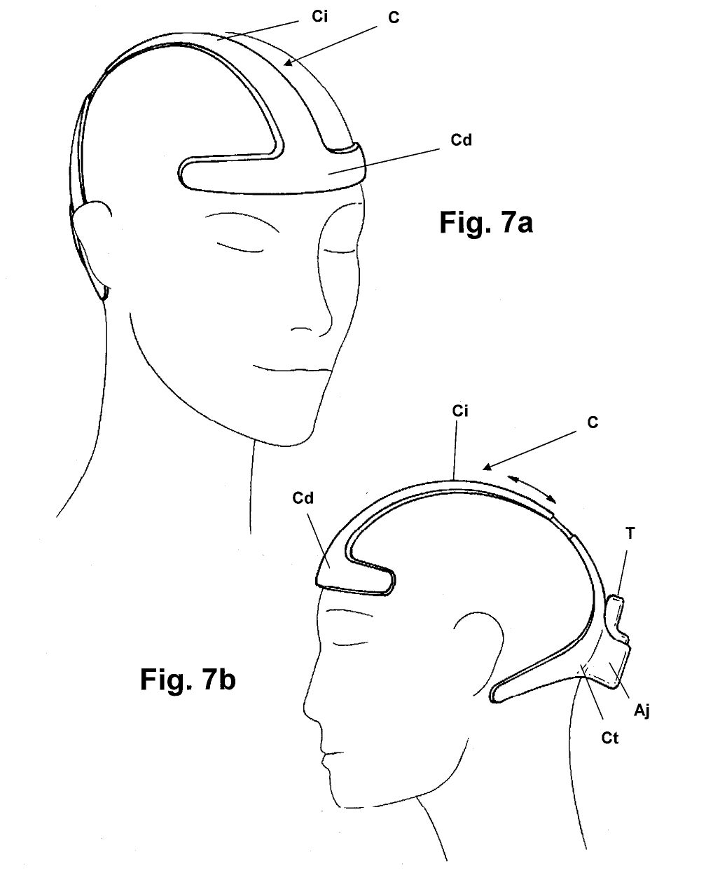

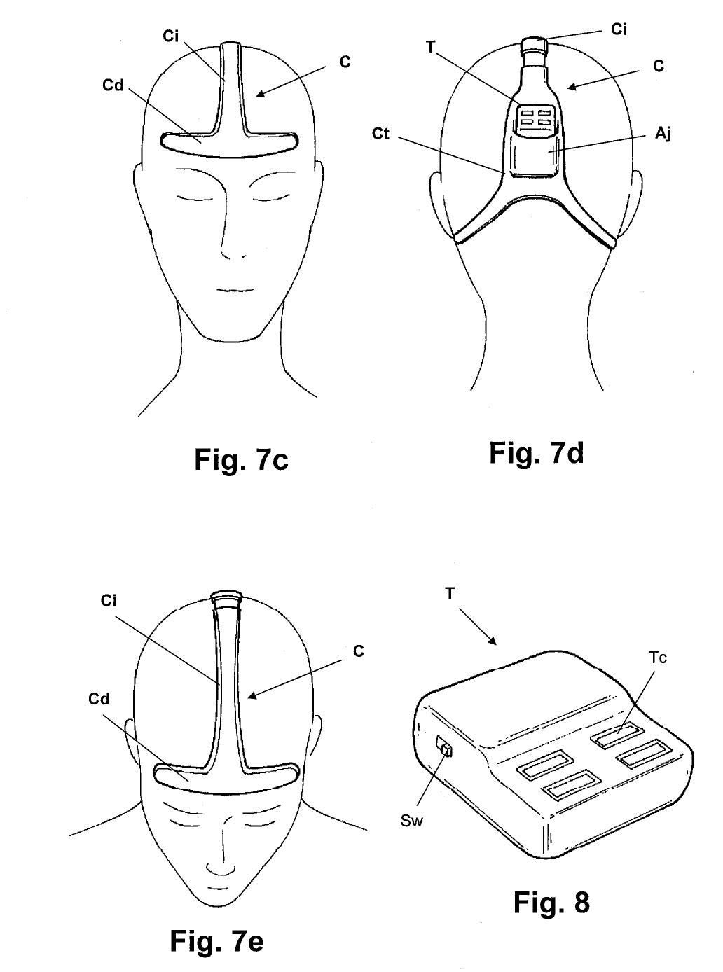

FIGS. 7a to 7e show a case for which (unlike that shown by FIGS. 4 and 5 referring to an ECG system) the biopotential signals to be conditioned are brain signals, for the embodiment of an encephalogram, in which the mentioned support C is coupled in the head of the patient.

For the embodiment shown in said FIGS. 7a to 7e the electrodes used are integrated in a front portion Cd of the support C, such that they are in contact with the forehead of the patient, although any other location which is considered suitable for conducting the ECG is also possible.

The intermediate portion Ci of the support C forms a strip Ci running along the head connecting the front portion Cd and a back portion Ct of the support C. Said strip Ci can be extended for the purpose of adapting the support C to different head sizes.

There is a housing Aj defined in the back portion Ct of the support C for the local electronic system SCM, which is in turned housed inside a case T, which is shown with greater detail in FIG. 8.

In the case T shown in said FIG. 8, a series of connectors Tc is observed for connecting the ends of corresponding cables (not shown) connected to the instrumentation amplifiers of the receiver electrodes, and to the voltage supplying electrode, and running along the strip Ci of the support C and are integrated therein.

Said case T also has on one of its sides a switch Sw for its handling by an operator for the purpose of activating/deactivating the local electronic system (SCM).

As has been indicated in a previous section, the present invention also relates, in a third aspect, to a method for controlling a weak electrical signal conditioning circuit which, although it is not limited to it, is applied to controlling a conditioning circuit according to the second aspect of the invention.

For the embodiments for which the method is applied to the proposed circuit according to the second aspect of the invention, specifically for the embodiments shown by FIGS. 4 and 5, the method comprises carrying out the actions already described in the description of the second aspect of the invention, including the generation, modification, adjustment and supply of the different signals Vref1, Vref2, Sc1, Sc2, Sg1 and Sg2, and monitoring the output signals VAmp1p, VAmp2p of the amplifiers Amp1, Amp2.

As has been previously described with reference to the circuit proposed by the second aspect of the invention, the method proposed by the third aspect also comprises carrying out said compensations performing the mentioned actions for generating, modifying, adjusting and supplying signals Vref1, Vref2, Se1, Sc2, Sg1 and Sg2, according to the ratio between the output signal of the instrumentation amplifier or amplifiers Amp1, Amp2 and the dynamic range thereof.

An embodiment of the method proposed by the third aspect of the invention is described below with reference to an instrumentation amplifier Amp1, although the second Amp2 or even other additional amplifiers are controlled in the same way as that explained below for Amp1.

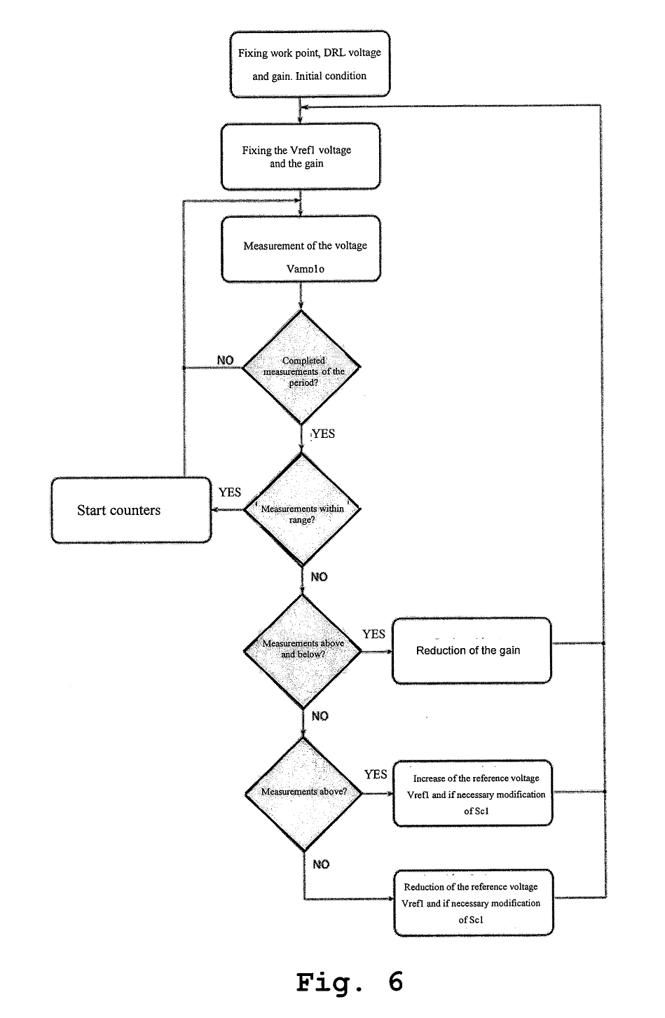

For said embodiment, shown by means of a flow chart in FIG. 6, the method proposed by the third aspect comprises carrying out the following steps, taking into account said ratio between the output signal of the instrumentation amplifier Amp1 and the dynamic range thereof:

a) fixing an initial work point which includes predetermining a value for the gain adjustment signal Sg1, according to the desired gain, and a substantially equal value for the reference electrical signal to be applied to the voltage supplying electrode E3 and for the reference voltage Vref1, i.e., equal to that generated by the constant voltage generating device D (see FIGS. 4 and 5).

Said step a) is shown in the first and second boxes (counting from the top) of the flow chart of FIG. 6.

b) monitoring the digitized output of the instrumentation amplifier Amp1 for a predetermined number of samples or period;

Step b) is shown in the third and fourth boxes of the flow chart of FIG. 6 (in which the monitored values are indicated as “measurements”). Said third and fourth boxes are connected by a return path (from the fourth to the third box) indicating that if the mentioned number of samples has not been reached, samples must continue to be acquired until it is so, and then the next box is entered into, i.e., the next step is carried out:

c) checking if the values of the signal VAmp1o obtained in said monitoring of said step b) are within the dynamic range of the instrumentation amplifier Amp1 (fifth box counting from the top), and:
if as a result of said step c) it is determined that the values of the monitored signal VAmp1o are within the dynamic range of the instrumentation amplifier Amp1, starting a series of relative counters at least to said number of samples or period and said monitored signal VAmp1o, and carrying out said steps b) and c) again;
if as a result of said step c) it is determined that the values of the monitored signal VAmp1o are outside the dynamic range of the instrumentation amplifier Amp1, passing to the sixth box counting from the top which refers to a dilemma, according to the response of which the method comprises alternatively carrying out the following steps:

d1) if there are values of the monitored signal VAmp1o above and below the dynamic range of the instrumentation amplifier Amp1, reducing the gain thereof by means of modifying said gain adjustment signal Sg1 (box to the right of the sixth box counting from the top) and its corresponding application (second box); and carrying out said steps b) and c) again;

d2) if there are only values of the monitored signal VAmp1o above the dynamic range of the instrumentation amplifier Amp1 (affirmative response to the dilemma of the seventh box counting from the top), at least increasing the value of the reference voltage Vref1 (box to the right of the seventh box counting from the top), applying it (second box), and carrying out said steps b) and c) again; or

d3) if there are only values of the monitored signal VAmp1o below the dynamic range of the instrumentation amplifier Amp1 (negative response to the dilemma of the seventh box counting from the top), at least decreasing the value of the reference voltage Vref1 (last box of the flow chart of FIG. 6 counting from the top), applying it (second box), and carrying out said steps b) and c) again.

As is indicated in the flow chart of FIG. 6, if it is necessary:

said step d2) further comprises modifying the direct current offset adjustment signal Sc1 and applying it to the direct current offset adjustment input DIGIN1 of the instrumentation amplifier Amp11, in order to reduce the direct current level in the monitored signal VAmp1o; and

said step d3) further comprises modifying the direct current offset adjustment signal Sc1 and applying it to the direct current offset adjustment input DIGIN1 of the instrumentation amplifier Amp11, in order to increase the direct current level in the monitored signal VAmp1o.

As has been previously mentioned the method proposed by the third aspect of the invention comprises compensating the direct current offsets experienced by other weak electrical signal or signals received by other receiver electrodes in contact with other areas of the patient H, in a manner similar to how the compensation with the weak electrical signal SE1 received by the first receiver electrode E1 is carried out.

A person skilled in the art could introduce changes and modifications in the embodiments described without departing from the scope of the invention as it is defined in the attached claims.

1. An electrophysiological sensor of the type which is based on conducting nanostructures which can transmit a weak electrical signal captured from the skin or from another part of an organic tissue to a transmitter means for transmitting said weak signal, said sensor comprising a plurality of said nanostructures which adopt a rigid filiform configuration and are chemically connected at one end to a conducting substrate electrically coupled to said transmitter means, being operable to at least partially penetrate said organic tissue or skin at their free end.

2. The electrophysiological sensor according to claim 1, wherein said plurality of nanostructures in the form of rigid filiform elements comprise conductive and inert carbon nanotubes grouped like bristles of a brush.

3. The electrophysiological sensor according to claim 1, wherein said carbon nanotubes have multiple walls.

4. The electrophysiological sensor according to claim 1, wherein said conducting substrate is made of highly doped silicon or titanium.

5. The electrophysiological sensor according to claim 1, wherein said nanotubes are at least partially coated with a coating facilitating a Red-Ox reaction.

6. The electrophysiological sensor according to claim 1, wherein said nanotubes are coated, in the region of their tips, which can come into contact with the skin, with a coating facilitating a Red-Ox reaction.

7. The electrophysiological sensor according to claim 1, wherein said nanotubes are at least partially coated with an Ag—AgCl coating.

8. The electrophysiological sensor according to claim 1, further comprising an envelopment casing housing said transmitter means for transmitting said weak signal which comprises a connector 4 which is continued in a conductor or wiring, the casing having arranged in an outer wall said conducting substrate supporting the nanostructures.

9. The electrophysiological sensor according to claim 1, further comprising a local amplifier in association with said transmitter means.

10. The electrophysiological sensor according to claim 1, adapted for incorporation in any of a plurality of different supports.

11. The electrophysiological sensor according to claim 10, wherein said conducting substrate is integrated in a material forming part of a surface of said garment or other article providing a substrate.

12. The electrophysiological sensor according to claim 1, wherein it further houses an electronic circuit for processing and treating the signal.

13. The electrophysiological sensor according to claim 1, further comprising a local amplifier and a digital electronic circuit for controlling amplification.

14. The electrophysiological sensor according to claim 1, further comprising a digital electronic circuit adapted for compressing the digitized signals prior to transmitting the signal.

15. The electrophysiological sensor according to claim 1, wherein it further houses a wireless electronic circuit for transmitting data.

16. The electrophysiological sensor according to claim 1, adapted for use in EEG, ECG, EMG, EOG, or for brain-machine interface applications, biometric applications or systems for detecting fatigue and hypovigilance.

17. The electrophysiological sensor according to claim 1, adapted for monitoring the wakefulness or sleep state in an individual.

18. A weak electrical signal conditioning circuit, for use with the electrophysiological sensor of claim 1 to 15, of the type said circuit comprising:
at least one adjustable-gain instrumentation amplifier with a high input impedance, with:
a first input in connection with a first receiver electrode in contact with a first area of a medium for receiving at least one of said weak electrical signals coming from said mediums, and
a second input in connection with a reference voltage,
a reference voltage generating device in connection with a voltage supplying electrode in contact with a second area of said medium for applying a reference electrical signal to it so that the voltage existing in the medium has a value substantially equal to that of said electrical signal generated by said reference voltage generating device,
compensation means for compensating at least the direct current offsets experienced by at least said weak electrical signal received by the first receiver electrode,
wherein said circuit is adapted for applying to the voltage supplying electrode, by means of said reference voltage generating device, a continuous electrical signal with a fixed value as said reference electrical signal, said reference voltage generating device being a constant voltage generating device, and said compensation means comprise at least:
a voltage generating device connected to said second input of said instrumentation amplifier, which is at least one in number, for generating and supplying to it an electrical signal with a reference voltage through said second input, for compensating at least the unwanted direct current offsets experienced by the weak electrical signal received by the first receiver electrode, and
a control system in connection with said voltage generating device and adapted for controlling it for the purpose of modifying the value of said reference voltage,
said compensation means being electrically insulated with respect to said constant voltage generating device and substantially with respect to said voltage supplying electrode for assuring that there is no current flow through the medium as a result of the action of the compensation means.

19. The circuit according to claim 18, wherein said compensation means are adapted for, for the purpose of complementing said compensation carried out by means of applying said reference voltage, supplying the instrumentation amplifier, through a direct current offset adjustment input, with a direct current compensation adjustment signal at the output of the instrumentation amplifier representative of a variable voltage value determined according to the direct current offset to be compensated experienced by at least said weak electrical signal received by the first receiver electrode.

20. The circuit according to claim 19, wherein said compensation means are also adapted for compensating alternating interfering signals by means of supplying said electrical signal with a reference voltage and/or said adjustment signal, to said second input and to said direct current offset adjustment input of the instrumentation amplifier, respectively.

21. The circuit according to claim 19, wherein said compensation means are adapted for, according to at least the ratio between the output signal of the instrumentation amplifier and the dynamic range thereof:
adjusting said gain of the instrumentation amplifier by means of sending a gain adjustment signal to a gain adjustment input thereof, and/or
carrying out said modification of the value of said reference voltage to be applied to the second input of the instrumentation amplifier, and/or
modifying the value of said variable voltage, and therefore said adjustment signal representative thereof to be applied to said direct current offset adjustment input of the instrumentation amplifier.

22. The circuit according to claim 21, wherein said gain adjustment input and said direct current offset adjustment input are one and the same input for programming the instrumentation amplifier, said adjustment signals being digital signals.

23. The circuit according to claim 18, wherein characterized in that it is applied to conditioning biopotential measurement signals, said medium being a patient, for the purpose of achieving a minimum current flow through the medium.

24. The circuit according to claim 23, wherein said voltage supplying electrode is in contact with a contact area of said patient, forming with said constant voltage generating device a right leg circuit for the purpose of achieving that the voltage of the medium is substantially equal to the voltage supplied by the constant voltage generating device.

25. The circuit according to claim 19, further comprising at least one second adjustable-gain instrumentation amplifier with a high input impedance, with:
a first input in connection with a second receiver electrode in contact with a third area of said medium for receiving at least another one of said weak electrical signals coming from the medium, and
a second input in connection with a second reference voltage,
said compensation means being adapted for also compensating at least the direct current offsets experienced by said weak electrical signal received by said second electrode,
said compensation means comprise a second voltage generating device connected to said second input of said second instrumentation amplifier, for generating and supplying to it an electrical signal with second reference voltage through said second input, for compensating at least the unwanted direct current offsets experienced by the weak electrical signal received by the second receiver electrode, and
said control system is also connected with said second voltage generating device, and adapted for controlling it for the purpose of modifying the value of said second reference voltage applied to the second input of the second instrumentation amplifier.

26. The circuit according to claim 25, wherein said compensation means are adapted for compensating direct current offsets experienced by the weak electrical signal received by said second electrode and alternating interfering signals, operating in a manner similar to how they operate with the instrumentation amplifier, including the supply of a respective direct current compensation adjustment signal and sending a gain adjustment signal to an adjustment input of same.

27. The circuit according to claim 18, further comprising a local electronic system including said control system, which is formed by at least one microcontroller or logic circuit, and said voltage generating devices, said control system being connected to the output of the instrumentation amplifier for monitoring it and operating accordingly.

28. The circuit according to claim 26, further comprising a local electronic system including said control system, which is formed by at least one microcontroller or logic circuit, and said voltage generating devices, said control system being connected to the outputs of the instrumentation amplifiers for monitoring them and operating accordingly.

29. The circuit according to claim 28, wherein said microcontroller or logic circuit comprises:
first and second outputs respectively connected to said first and second voltage generating devices, which are respective digital-to-analog converters, the outputs of which are respectively connected to the second input of the first instrumentation amplifier and to the second input of the second instrumentation amplifier, for sending it said electrical signals with reference voltage after their conversion to an analog format, and
third and fourth outputs respectively connected to said adjustment inputs of the instrumentation amplifiers, for supplying them with said adjustment signals in digital format.

30. The circuit according to claim 29, wherein at least said instrumentation amplifiers and associated circuitry are supported by a support which also supports at least one of said electrodes.

31. The circuit according to claim 30, wherein at least part of said local electronic system is also supported by said support.

32. The circuit according to claim 31, wherein said local electronic system comprises a communications module adapted for wirelessly communicating with a remote control system.

33. The circuit according to claim 32, wherein said communications module is adapted for communicating with said remote control system in a two-way manner.

34. The circuit according to claim 33, wherein said remote control system is adapted for wirelessly receiving, from the local electronic system, the digital values representative of the output signal of the instrumentation amplifier or amplifiers, and for analyzing them.

35. The circuit according to claim 34, wherein said remote control system is adapted for carrying out at least part of:
said adjustment of said gain of the instrumentation amplifier or amplifiers, and/or
said modification of the value of said reference voltage or voltages to be applied to the second input of the instrumentation amplifier or amplifiers, and/or
said modification of said adjustment signal or signals to be applied to said direct current offset adjustment input of the instrumentation amplifier or amplifiers, and
for carrying out the corresponding sending of the digital values of adjustment signals and/or of reference voltages to the local electronic system.

36. The circuit according to claim 28, wherein said local electronic system is adapted for carrying out:
said adjustment of said gain of the instrumentation amplifiers, and/or
said modification of the value of said reference voltages to be applied to the second input of the instrumentation amplifiers, and/or
said modification of said adjustment signals to be applied to said direct current offset adjustment inputs of the instrumentation amplifiers.

37. The circuit according to claim 23, wherein at least one of said electrodes comprises at least one electrophysiological sensor of the type which is based on conducting nanostructures which can transmit a biopotential electrical signal captured from the skin or from another part of an organic tissue of said patient to a transmitter means for transmitting said signal, and which comprises a plurality of said nanostructures which adopt a rigid filiform configuration and are chemically connected at one end to a conducting substrate electrically coupled to said transmitter means, being operable to at least partially penetrate said organic tissue or skin of said patient at their free end.

38. A method for controlling a weak electrical signal conditioning circuit, comprising:
receiving at least one of said weak electrical signals coming from a medium, through a first receiver electrode in contact with a first area of said medium,
sending said received weak electrical signal to a first input of an adjustable-gain instrumentation amplifier with a high input impedance,
applying a reference voltage to a second input of said instrumentation amplifier,
applying a reference electrical signal to a voltage supplying electrode in contact with a second area of said medium so that the voltage existing in the medium has a value substantially equal to that of said reference electrical signal applied to said voltage supplying electrode,
compensating at least the direct current offsets experienced by at least said weak electrical signal received by the first receiver electrode,
wherein:
said reference electrical signal applied to the voltage supplying electrode is a continuous electrical signal with a fixed value, independent from the common-mode signal of said instrumentation amplifier, and
in that said compensation of at least the direct current offsets experienced by at least said weak electrical signal received by the first receiver electrode, is automatically carried out by means of compensation means substantially electrically insulated with respect to said voltage supplying electrode for assuring that there is no current flow through the medium as a result of the action of the compensation means.

39. The method according to claim 38, further comprising carrying out said compensation of at least the direct current offsets experienced by at least said weak electrical signal received by the first receiver electrode, by means of applying said reference voltage and modifying its value in a controlled manner.

40. The method according to claim 39, further comprising complementing said compensation carried out by means of applying said reference voltage, by means of supplying the instrumentation amplifier, through a direct current offset adjustment input, with a direct current compensation adjustment signal at the output of the instrumentation amplifier representative of a variable voltage value determined according to the direct current offset to be compensated experienced by at least said weak electrical signal received by the first receiver electrode.

41. The method according to claim 40, further comprising compensating alternating interfering signals by means of supplying said electrical signal with a reference voltage and/or said adjustment signal, to said second input and to said direct current offset adjustment input of the instrumentation amplifier, respectively.

42. The method according to claim 39, further comprising carrying out said compensation or compensations for compensating the common-mode voltage variations of at least said instrumentation amplifier.

43. The method according to claim 40, further comprising carrying out said compensations according to at least the ratio between the output signal of the instrumentation amplifier and the dynamic range thereof, by means of carrying out at least one of the following actions, or a combination thereof:
adjusting said gain of the instrumentation amplifier by means of sending a gain adjustment signal to a gain adjustment input thereof;
carrying out said modification of the value of said reference voltage to be applied to the second input of the instrumentation amplifier;
modifying the value of said variable voltage, and therefore said adjustment signal representative thereof to be applied to said direct current offset adjustment input of the instrumentation amplifier.

44. The method according to claim 43, further comprising carrying out the following steps:
a) fixing an initial work point which includes predetermining a value for said gain adjustment signal, according to the desired gain, and a substantially equal value for said reference electrical signal to be applied to the voltage supplying electrode and for said reference voltage to be applied to the second input of the instrumentation amplifier;
b) monitoring the output of the instrumentation amplifier for a predetermined number of samples or period;
c) checking if the values of the signal obtained in said monitoring of said step b) are within the dynamic range of the instrumentation amplifier, and:
if as a result of said step c) it is determined that the values of the monitored signal are within the dynamic range of the instrumentation amplifier, starting a series of counters relative to at least said number of samples or period and said monitored signal, and carrying out said steps b) and c) again;
if as a result of said step c) it is determined that the values of the monitored signal are outside the dynamic range of the instrumentation amplifier, alternatively carrying out the following steps:
d1) if there are values of the monitored signal above and below the dynamic range of the instrumentation amplifier, reducing the gain thereof by means of modifying and applying said gain adjustment signal; and carrying out said steps b) and c) again;
d2) if there are only values of the monitored signal above the dynamic range of the instrumentation amplifier, at least increasing the value of said reference voltage to be applied to the second input of the instrumentation amplifier, applying it, and carrying out said steps b) and c) again; or
d3) if there are only values of the monitored signal below the dynamic range of the instrumentation amplifier, at least decreasing the value of said reference voltage to be applied to the second input of the instrumentation amplifier, applying it, and carrying out said steps b) and c) again.

45. The method according to claim 44, wherein:
said step d2) further comprises modifying said direct current offset adjustment signal and applying it to said direct current offset adjustment input of the instrumentation amplifier, in order to reduce the direct current level in the monitored signal; and
said step d3) further comprises modifying said direct current offset adjustment signal and applying it to said direct current offset adjustment input of the instrumentation amplifier, in order to increase the direct current level in the monitored signal.

46. The method according to claim 38, further comprising compensating at least the direct current offsets experienced by another weak electrical signal received by a second receiver electrode in contact with a third area of said medium, in a manner similar to how the compensation is carried out with said weak electrical signal received by said first receiver electrode.

47. The method according to claim 38, wherein the method is applied to conditioning biopotential measurement signals, said medium being a patient, for the purpose of achieving a minimum current flow through the medium.

48. The electrophysiological sensor according to claim 10, wherein said plurality of different supports are selected from the group consisting of garments, pillows, and/or mattresses.
